(19) United States
(12) Patent Application Publication
(10) Pub. No.: US 2021/0327598 A1
(43) Pub. Date: Oct. 21, 2021

(54) AUTOMATED DATA VERIFICATION

(71) Applicant: Clover Health, Jersey City, NJ (US)

(72) Inventors: Jasmine Tsai, Jersey City, NJ (US); Rohan Gupta, San Francisco, CA (US); Christopher Paul Hartfield, San Francisco, CA (US)

(21) Appl. No.: 16/853,328

(22) Filed: Apr. 20, 2020

(51) Int. Cl. G16H 80/00; G16H 50/20; G06Q 40/08; G06Q 50/22; G06N 7/00

(52) U.S. Cl. CPC G16H 80/00; G16H 50/20; G06N 7/005; G06Q 50/22; G06Q 40/08

(57) ABSTRACT

Techniques are described herein for automatically verifying medical provider data. A service provider may receive medical provider data from one or more data sources. The data sources may include websites associated with health care services, search engines, crowd-sourced reviews, insurance claims from medical providers, feedback from patients (e.g., members), and the like. The service provider may compare the received medical provider data to stored medical provider data in a datastore, such as that stored in a medical provider account. The service provider may determine a probability that the stored medical provider data is accurate. Based on a determination that the probability is above a threshold, the service provider may automatically verify the stored medical provider data. Based on a determination that the probability is below the threshold, the service provider may request manual verification of the stored medical provider data.

### BACKGROUND

Health insurance carriers provide members with a network of healthcare professionals contracted to provide services to the members at discounted rates. The health insurance carriers may provide the members with information about the healthcare professionals, to enable the members to make informed decisions about which healthcare professional to choose. The information may include medical specialties, locations for services, contact information, and the like. Accuracy of the information may be paramount to the member when choosing a health care professional. Traditionally, health insurance carriers manually verify and update medical provider data. However, the manual verification and updating of the provider data is cumbersome and labor intensive. As such, the provider data may not be updated in a timely manner, thereby resulting in mis-information leading to a frustrating experience for members in choosing a healthcare professional.

### BRIEF DESCRIPTION OF THE DRAWINGS

The detailed description is described with reference to the accompanying figures. In the figures, the left-most digit(s) of a reference number identifies the figure in which the reference number first appears. The use of the same reference numbers in different figures indicates similar or identical components or features.

FIG. 1 is a schematic view of an example system usable to automatically verify medical provider data based on data from multiple sources, as described herein.

FIG. 2 illustrates a block diagram of example components associated with a data verification system.

FIG. 3 illustrates a block diagram illustrating an example system of computing devices usable to implement example techniques described herein.

FIG. 4 illustrates an example process for automatically verifying medical provider data, utilizing the techniques described herein.

FIG. 5 illustrates an example process for automatically verifying a medical provider specialty based on received insurance claims, utilizing the techniques described herein.

FIG. 6 illustrates an example process for automatically verifying a medical provider quality metric, utilizing the techniques described herein.

### DETAILED DESCRIPTION

This application describes techniques for automatically verifying provider data associated with healthcare professionals (e.g., medical providers). For instance, a service provider computing device may maintain a database of provider data (e.g., provider database) associated with medical providers. The service provider computing device may access one or more data sources that publish provider data associated with the medical providers. The data source(s) may include healthcare databases, search engine databases, crowd-sourced review databases, and the like. The service provider computing device may compare the provider data stored in the provider database to the provider data published by the data sources(s) and may determine a probability that the provider data stored in the provider database is accurate. Based on the probability, the service provider computing device may either verify the provider data or provide an indication that the provider data may be inaccurate.

In various examples, the service provider computing device may determine to verify provider data associated with the medical providers stored a provider database. In some examples, the verification may be performed periodically (e.g., weekly, monthly, quarterly, etc.) and/or at non-periodic intervals (e.g., random timing). In some examples, the verification may be performed continuously.

In some examples, based on a determination to verify the provider data, the service provider computing device may access one or more data sources. The data source(s) may include databases comprising provider data, such as those accessible via a website. In various examples, the data source(s) may include sources associated with providing medical provider data, such as, for example, the Council for Affordable Quality Healthcare (CAQH), the National Committee for Quality Assurance (NCQA), National Plan and Provider Enumeration System (NPPES) National Provider Identifier (NPI) database, and the like. In some examples, the data source(s) may include sources associated with providing other services, such as location related services (e.g., web-based mapping applications), quality review services (e.g., crowd-sourced review forums, etc.), business directory services, and the like.

In various examples, the service provider computing device may determine a confidence score associated with the data provided by the data source(s). In some examples, the confidence score may apply to all data provided by the data source. In such examples, a data source may have associated therewith a confidence score corresponding to the information provided by the data source. For example, a first data source may be updated often and therefore may have a high confidence score (e.g., 0.75, 0.8, etc.) whereas a second data source may not be updated as frequently and may have associated therewith a lower confidence score (e.g., 0.50, 0.60, etc.).

In some examples, the confidence score may apply to each data set provided by the data source. A data set may include a type of data provided by the data source, such as name of a medical provider, NPI, address, phone number, website, specialty, credentials (e.g., certifications), type of medical practice affiliation (e.g., solo practice, group practice, etc.), name of affiliated medical practice, hours of operations, and the like. For example, a first data source associated with an NPI issuer may be known to provide accurate data with respect to medical provider NPIs but may not often update addresses associated with the medical providers. The first data source may have associated therewith a high confidence score with respect to NPIs (e.g., 0.8, 0.3, 0.35, etc.) and a lower confidence score with respect to addresses (e.g., 0.3, 0.4, etc.).

In some examples, the service provider computing device may determine the confidence score with respect to the data source and/or data set provided by the data source based on experiments performed on the data, such as manual verifications of data provided by the data source. In some examples, the service provider computing device may apply a heuristic technique to determine the confidence score with respect to the data source and/or data set provided by the data source. For example, governmental data sources may include a higher confidence score with respect to credentialing and/or identifiers (e.g., NPIs, etc.) and a mapping application may include a higher confidence score with respect to location data and/or other contact information.

In various examples, the service provider computing device compare provider data received from the data source(s) to provider data stored in the provider datastore. In some examples, the service provider computing device may determine a probability of accuracy associated with provider data stored in the provider datastore. The probability of accuracy associated with the provider data may based on the confidence scores associated with the data source(s) and/or the data sets corresponding thereto. In various examples, the probability of accuracy may be based on a comparison of similar data sets across two or more data sources and/or compared to the provider data stored in the provider database of the service provider computing device. For example, a first address associated with a medical provider published by a data source may be compared to a second address associated with the medical provider stored in the provider database. Based on a determination that the first address and the second address match, the service provider computing device may determine a high probability of accuracy associated with the address stored in the provider database. Based on a determination that the first address and the second address do not match, the service provider computing device may determine the probability of accuracy of the address stored in the provider datastore based on the confidence scores associated with the data source and/or the data sets associated therewith.

In some examples, based on a determination that the probability of accuracy is at or below a threshold probability, the service provider computing device may access an additional data source to determine an updated probability of accuracy. In some examples, the additional data source may be accessed based on a confidence score associated therewith being above a threshold confidence score. For example, the service provider computing device may compare a phone number associated with a medical provider stored in a provider database to a phone number associated with the medical provider published via a first data source. Based on a determination that the phone numbers do not match, the service provider computing device may determine the probability of accuracy is below a threshold probability. The service provider computing device may then access provider data via a second data source with a confidence score above a threshold (e.g., overall confidence score, confidence score associated with phone numbers, contact information, etc.). Based on a comparison of the phone number stored in the provider database, the phone number published in the first data source, and/or the phone number published in the second data source, the service provider computing device may determine the updated probability of accuracy.

In some examples, based on a determination that the probability of accuracy is at or below a threshold probability, the service provider computing device may determine to request a manual verification of the provider data associated with the provider datastore. In some examples, the service provider computing device may surface an instruction to manually verify the provider data. In such examples, the instruction may include the particular type of data to be verified (e.g., address, phone number, website, NPI, specialty, etc.), a medical provider associated with the data (e.g., name, identifier, etc.), information regarding the data source (e.g., name of the data source, confidence scores, etc.), a date and/or time the data was last verified, and/or any other information to assist in data verification.

Based at least in part on the instruction surfaced to manually verify the provider data (e.g., a verification request associated with provider data), a human may intervene to verify the provider data. The verification may include a manual verification of and/or update to the provider data. In some examples, the update may include a date/time associated therewith, data associated with one or more resources used to verify and/or update the data, and/or any other information relevant to the verification and/or update of the data.

In some examples, based on a determination that the probability of accuracy is at or below the threshold probability and the confidence score associated with the data source is high (e.g., above a threshold confidence score), the service provider computing device may automatically update the provider data associated with the provider database. In such examples, the provider database may be automatically updated to ensure current information is available to members (e.g., members associated with an insurance service provider, patients) and/or medical providers accessing the provider database. For example, the service provider computing device may determine that an address associated with a medical provider published via a mapping application does not match the address associated with the medical provider stored in a provider datastore. Based on a determination that the mapping application has a 35% accuracy with respect to addresses, the service provider computing device may assign a high confidence (e.g., 0.3, 0.35, 0.38, etc.) to the address published by the mapping application. Based on the high confidence, the service provider computing device may automatically update the address in the provider database.

In various examples, based on a determination that a probability of accuracy meets or exceeds a threshold probability, the service provider computing device may automatically verify the provider data associated with the provider datastore, such as without human intervention. In such examples, the provider data stored in the provider database may be automatically verified to ensure accurate information is available to members and/or medical providers accessing the provider database.

The techniques described herein improve database management, and specifically data verification, by automatically verifying and/or updating medical provider data stored in a provider database. Traditionally, the provider data stored in a provider database has been manually verified and/or updated. Often, insurance providers managing the provider database hire hundreds of employees to periodically verify and/or update provider data stored in the provider database. Thus, at least because the techniques described herein automate a data verification and/or update process previously performed by humans, the techniques described herein improve database management and/or data verification.

Additionally, the techniques described herein improve performance of one or more computing devices by not accessing and/or pulling data from a source known to be unreliable. For instance, the service computing device may determine a confidence score associated with a data source and/or associated with data sets corresponding to the data source. Based on a determination that a confidence score associated with the data source and/or a data set associated therewith is below a threshold confidence score, the service provider computing device may not access the data source and/or pull data published thereby for data verification. By not pulling and/or storing data known to be unreliable, the service provider computing device may increase an amount of storage space available for other, more reliable data. Furthermore, by not attempting to access and/or pull data from unreliable sources, the service provider computing device may have additional processing power available for other computing tasks, such as pulling data from reliable sources, computing probabilities of accuracy, and the like.

These and other aspects are described further below with reference to the accompanying drawings. The drawings are merely example implementations and should not be construed to limit the scope of the claims. For example, while examples are described as relating to medical provider data, the techniques may be implemented may be used to automate verifications and/or updates of data stored in a database including any type of data, such as member data, client data, subscriber data, business data, and the like.

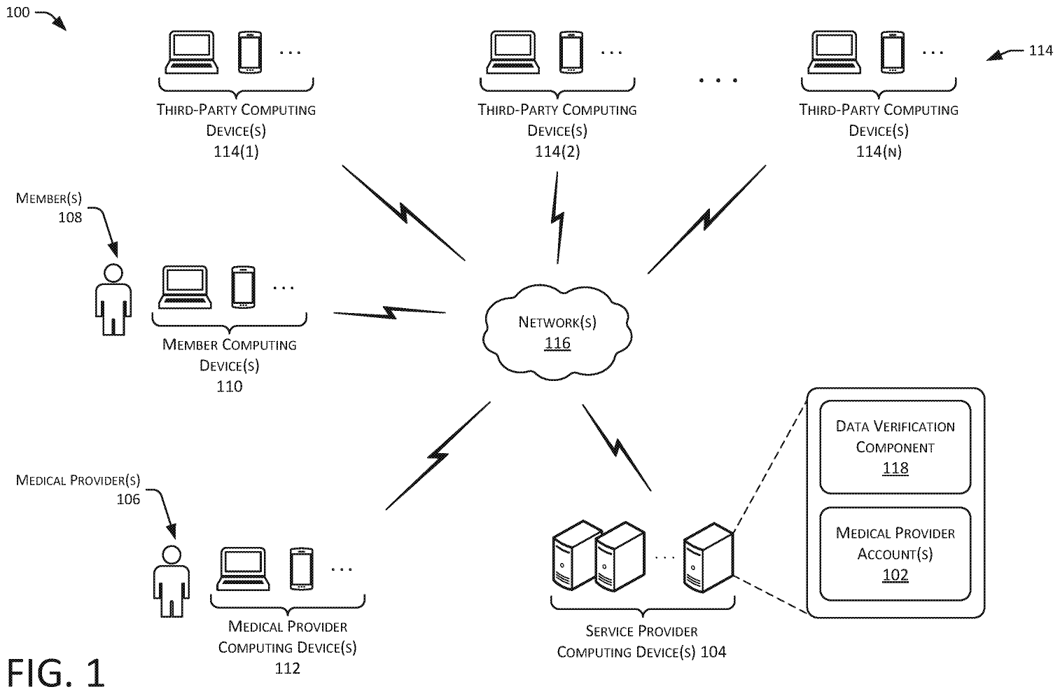

FIG. 1 is a schematic view of an example system 100 usable to implement the techniques described herein to automatically verify provider data stored in one or more medical provider accounts 102 based on data provided by multiple remote sources, via the system 100. In some examples, the system 100 may include a one or more service provider computing devices 104 (e.g., service provider 104) configured to manage a database comprising the medical provider account(s) 102, such as to provide medical provider data associated with one or more medical providers 106 to one or more members 108 associated with the service provider 104. In some examples, the member(s) 108 may access the medical provider data via one or more member computing devices 110.

In at least one example, the service provider computing device(s) 104 may be associated with providing medical insurance services to the member(s) 108 (e.g., patients) associated therewith. In such an example, the service provider 104 may provide medical provider data to members 108 for identifying and locating medical providers 106. Additionally, the medical providers 106 may access medical provider data via one or more medical provider computing device(s) 112, such as for providing a referral for a member 108 to visit another medical provider 106. In various examples, the service provider 104 may additionally be configured to receive claims from medical provider(s) 106 and/or member(s) 108, such as via respective devices 112 and 110, and/or provide other services such as providing member data (e.g., health history, treatment history, medical records, etc.) to the medical provider(s) 106, such as via one or more medical provider computing devices 112.

In various examples, the service provider computing device 104 may store the medical provider data in the medical provider account(s) 102, such as in a database formatted based at least in part on the medical provider(s) 106 (e.g., medical provider database). In some examples, the medical provider data may be determined based on data submitted by the medical provider(s) 106, such as in establishing an account with the service provider 104, and/or data received from one or more third-party computing devices 114.

Each of the medical provider device(s) 106, the member device(s) 110, and the third-party computing device(s) 114 may be remote from the service provider 104 and may include one or more processors and memory storing computer executable instructions to implement the functionality discussed herein attributable to the respective computing devices. In some examples, the medical provider device(s) 106, the member device(s) 110, and the third-party computing device(s) 114 may include desktop computers, laptop computers, tablet computers, mobile devices (e.g., smart phones or other cellular or mobile phones, mobile gaming devices, portable media devices, etc.), or other suitable computing devices. The medical provider device(s) 106, the member device(s) 110, and the third-party computing device(s) 114 may execute one or more client applications, such as a web browser (e.g., Microsoft Windows Internet Explorer, Mozilla Firefox, Apple Safari, Google Chrome, Opera, etc.) or a native or special-purpose client application (e.g., service provider application, etc.), to access and view information provided by the service provider computing device(s) 104 over a network 116.

Network 116 may represent a network or collection of networks (such as the Internet, a corporate intranet, a virtual private network (VPN), a local area network (LAN), a wireless local area network (WLAN), a cellular network, a wide area network (WAN), a metropolitan area network (MAN), or a combination of two or more such networks) over which the medical provider device(s) 106, the member device(s) 110, and the third-party computing device(s) 114 may access the service provider computing device(s) 104 and/or communicate with one another.

The service provider computing device(s) 104 may include one or more servers or other computing devices, any or all of which may include one or more processors and memory storing computer executable instructions to implement the functionality discussed herein attributable to the social networking system or digital platform. The service provider computing device(s) 104 may store data associated with the medical provider(s) 106 (e.g., medical provider data), such as the medical provider account(s) 102. The medical provider data may include provider location information (e.g., office locations, home address of medical provider, etc.), names and credentials of medical providers associated with a medical office and/or location, type of medical practice affiliation (e.g., solo practice, group practice, etc.), contact information (e.g., address, phone number, website, electronic mail, etc.), clinical visit times (e.g., average time, preferred (target) time, longest visit, shortest visit, etc.), insurance billing history, procedural history, procedural and/or practice specialties, provider quality metric (e.g., based on quality of service (e.g., based on feedback from members, professional organizations, awards earned, etc.), claim submissions (e.g., submitted on time with limited errors, etc.), percentage of patients associated with the service provider, use and management of the service provider application (e.g., clinical assessments, submitting referrals, etc.), ease of scheduling, delays associated with scheduling, etc.), and other information associated with the medical provider.

As illustrated in FIG. 1, the service provider computing device(s) 104 may include a data verification component 118 configured to automatically verify medical provider data stored in the medical provider account(s) 102. In various examples, the data verification component 118 may be configured to automatically update the medical provider data. In some examples, the verification and/or update may be performed periodically (e.g., weekly, monthly, quarterly, etc.) and/or at non-periodic intervals (e.g., random timing). In some examples, the verification and/or update may be performed continuously. In such examples, the service provider computing device(s) 104 may continually search data sources, such as those associated with the third-party computing device(s) 114 to verify medical provider data.

In various examples, the data verification component 118 may access medical provider data stored on one or more third-party computing devices 114, such as third-party computing devices 114(1), 114(2), and 114(N), via the network 116. The third-party computing device(s) 114 may include data sources configured to publish or otherwise provide various types of data. The third-party computing device(s) 114 may include devices associated with providing medical provider data, such as websites associated with the Council for Affordable Quality Healthcare (CAQH), the National Committee for Quality Assurance (NCQA), National Plan and Provider Enumeration System (NPPES) National Provider Identifier (NPI) database, and the like. In some examples, the third-party computing device(s) 114 may include websites associated with providing other services, such as location related services (e.g., web-based mapping applications), quality review services (e.g., crowd-sourced review forums, etc.), business directory services, and the like.

In various examples, the data verification component 118 may determine a confidence score associated with each third-party computing device 114(1), 114(2), and 114(n) as a data source. In such examples, the confidence score may include a confidence the service provider 104 has in each third-party computing device 114(1), 114(2), and 114(n) to provide accurate data. In various examples, the data verification component 118 may determine a confidence score associated with particular data provided by each third-party computing device 114(1), 114(2), and 114(n). In such examples, the data verification component 118 may determine a first confidence score for a first type of medical provider data, a second confidence score for a second type of medical provider data, and so on. For example, the data verification component 118 may determine that a first third-party computing device 114(1) consistently provides accurate data with respect to medical provider addresses but does not provide accurate information with respect to medical provider telephone numbers. Accordingly, the data verification component 118 may assign a confidence of 0.8 to the addresses and 0.5 to the phone numbers provided by the third-party computing device 114(1). Though these are merely illustrative examples, and any other values of confidence scores are contemplated herein.

In various examples, the confidence scores in the third-party computing device(s) 114 as sources of accurate data (e.g., overall confidence score) and/or as sources of accurate types of medical provider data (e.g., addresses, phone numbers, NPIs, credentials, specialties, etc.) may be determined based on experiments performed on the data, such as manual verifications of data provided by third-party computing device(s) 114. In some examples, the data verification component 118 may apply a heuristic technique to determine the confidence score with respect to a third-party computing device(s) 114, such as third-party computing device 114(1) and/or particular types of data provided by the third-party computing device 114(1).

In various examples, the data verification component 118 may access medical provider data from the third-party computing device(s) 114 via the network, such as by accessing a website including the medical provider data. In some examples, the data verification component 118 may receive the medical provider data from the third-party computing device(s) 114, such as by downloading the medical provider data stored on the third-party computing device(s) 114.

In some examples, the data verification component 118 may determine one or more third-party computing device(s) 114 to access for medical provider data and/or types of medical provider data based in part on the confidence scores associated therewith. In some examples, the data verification component 118 may access third-party computing device(s) 114 that have a confidence score above a first threshold confidence score. The threshold confidence score may include an overall confidence score associated with the third-party computing device(s) 114. For example, a first third-party computing device 114(1) may have an overall confidence score below the first threshold value and a second third-party computing device 114(2) may have an overall confidence score above the first threshold confidence score. The data verification component 118 may determine to access the second third-party computing device 114(2) to verify medical provider data.

In some examples, the data verification component 118 may determine a third-party computing device 114 to access for data verification of a type of data (e.g., data set, addresses, credentials, etc.) based on a confidence score associated with the type of data as provided by the third-party computing device 114. In some examples, the data verification component 118 may access the third-party computing device 114 based on a determination that the confidence score associated with the type of data is above a threshold confidence score. In some examples, the threshold confidence score may be associated with the type of data. In such examples, different type of data may have different threshold confidence scores associated therewith. For example, a third-party computing device 114(N) may have associated therewith a first confidence score associated with addresses and a second confidence score associated with phone numbers. The first confidence score may be above a threshold confidence score associated with addresses and the second confidence score may be below a threshold confidence score associated with phone numbers. The data verification component 118 may access the third-party computing device 114(N) to verify addresses associated with medical providers.

In various examples, the data verification component 118 may compare data stored in the medical provider account(s) 102 against medical provider data accessed via the third-party computing device(s) 114. The data verification component 118 may determine a probability of accuracy associated with medical provider data stored in the medical provider account(s) 102 based on the comparison. For example, the data verification component 118 may determine that a phone number stored in a medical provider account 102 is the same as a phone number published by a first third-party computing device 114(1) and a second third-party computing device 114(2), and an address stored in the medical provider account 102 is different from the address published by the first third-party computing device and the same as the address published by the second third-party computing device. The data verification component 118 may assign a probability of accuracy of 100% to the phone number and a probability of 50% to the address based on the comparisons of medical provider data.

In some examples, the probability of accuracy associated with the provider data may be based on the confidence scores associated with the third-party computing device(s) 114 (e.g., overall confidence score, confidence score associated with types of data). For example, the data verification component 118 may determine to verify an address associated with medical providers 106 and may access a third-party computing device 114, such as third-party computing device 114(1) with an associated address confidence score above a threshold value. The data verification component 118 may compare a first address associated with a medical provider 106 published by the third-party computing device 114(1) may be compared to a second address associated with the medical provider stored in a medical provider account 102 associated therewith. Based on a determination that the first address and the second address match, the data verification component 118 may determine a high probability of accuracy (e.g., 85%, 30%, etc.) associated with the address stored in the medical provider account 102, at least based on the address confidence score being above a threshold value.

In various examples, the data verification component 118 may be configured to access a first third-party computing device 114(1), such as that with a high confidence score (e.g., overall and/or associated with a particular type of data above a threshold) to verify data stored in the medical provider account(s) 102. In such examples, the data verification component 118 may be configured to verify data with a high degree of accuracy based on the high confidence score associated with the first third-party computing device 114(1). In some examples, the data verification component 118 may access additional third-party computing device 114 based on a discrepancy identified (e.g., difference in provider data) between the first third-party computing device 114 and the data stored in the medical provider account(s) 102. Continuing the example from above, based on a determination that the first address and the second address do not match, the data verification component 118 may access a second third-party computing device 114(2) and/or a third third-party computing device 114(N) to determine the probability of accuracy of the address stored in the medical provider account 102.

In some examples, the data verification component 118 may access additional third-party computing device 114 based on a determination that the probability of accuracy associated with medical provider data is below a threshold probability (e.g., 50%, 60%, 75%, etc.). In some examples, the data verification component 118 may access first medical provider data via the first third-party computing device 114(1). The first medical provider data may include a type of medical provider data. The data verification component 118 may compare the first medical provider data to medical provider data stored in the medical provider account 102 and may determine that a probability of accuracy in the medical provider data stored in the medical provider account 102 is below the threshold probability. The data verification component 118 may access a second third-party computing device 114(2) to update the probability of accuracy associated with the medical provider account 102.

Additionally or alternatively, the probability of accuracy associated with the medical provider account 102 may be determined based on medical provider data received from the medical provider computing device(s) 112 via the network. In some examples, the medical provider 106 may send updated medical provider data to the service provider 104, such as based on a change to the medical provider data. In such examples, the data verification component 118 may determine a current probability of accuracy associated with the medical provider data is below a threshold probability and may update the medical provider account 102 associated with the medical provider 106.

In some examples, the medical provider 106 may submit claims for services rendered to one or more members 108 to the service provider 104. The data verification component 118 may determine medical provider data included in the claims. The data verification component 118 may compare the medical provider data included in the claims to the medical provider data stored in an associated medical provider account 102 to determine the probability of accuracy of the data stored in the medical provider account 102. For example, the claims submitted by the medical provider may include a different phone number than a phone number stored in the medical provider account. The data verification component 118 may determine, based on the determined difference in phone numbers, that the probability of accuracy associated with the stored phone number is 50%, though this is merely an illustrative example probability and any other lower or higher probability is contemplated herein.

In various examples, the data verification component 118 may be configured to verify a specialty associated with the medical provider 106, such as that stored in a medical provider account 102, based in part on the submitted claims. In such examples, the data verification component 118 may determine a difference between the specialty associated with the medical provider 106 stored in the medical provider account 102 and services rendered by the medical provider 106 based on the submitted claims. In some examples, the probability of accuracy associated with the specialty may be determined based on a percentage of claims submitted for services related to the specialty. In some examples, the probability of accuracy may be based on the percentage of claims being above a threshold percentage of claims. For example, a data verification component 118 may determine that 30% of the claims submitted by a medical provider with an associated specialty of cardiology 30% are for angioplasties. As such, data verification component 118 may determine that the probability of accuracy associated with the cardiology specialty is 30% or higher. For another example, the data verification component 118 may determine that 60% of the claims submitted by the cardiologist are for general practitioner-related services. As such, the data verification component 118 may determine that the probability of accuracy associated with the cardiology specialty is 70%.

In some examples, based on a determination that the probability of accuracy is at or below the threshold probability, the data verification component 118 may determine to request a manual verification of the provider data associated with the medical provider account(s) 102. In some examples, the data verification component 118 may provide an indication that verification is requested. In such examples, the indication may include the particular type of data to be verified (e.g., address, phone number, website, NPI, specialty, etc.), a medical provider associated with the data (e.g., name, identifier, etc.), information regarding the third-party computing device corresponding to the disparate medical provider data (e.g., name of the data source, associated website, confidence scores, etc.), a date and/or time the medical provider data was last verified, and/or any other information to assist in data verification. In some examples, the indication may include an instruction to verify the medical provider data. In such examples, the instruction may be surfaced on a display of a service provider computing device 104.

In various examples, based on the indication that verification is requested (e.g., a verification request associated with medical provider data), a human may intervene to verify the medical provider data associated with the medical provider account(s) 102. The verification may include a manual verification of and/or update to the medical provider data. In some examples, the update may include a date/time associated therewith, data associated with one or more resources used to verify and/or update the data, and/or any other information relevant to the verification and/or update of the data.

In some examples, based on a determination that the probability of accuracy is at or below the threshold probability and that confidence scores associated with the third-party computing device(s) 114 are above a threshold confidence score, the data verification component 118 may automatically update the provider data associated with the medical provider accounts 102, such as without human intervention. In such examples, the medical provider accounts 102 may be automatically updated to ensure current information is available to members 108 and/or medical providers 106 accessing the medical provider accounts 102. For example, the data verification component 118 may determine that an address associated with a medical provider 106 published via a mapping application does not match the address associated with the medical provider 106 stored in a medical provider account 102. Based on a determination that the mapping application has a 35% accuracy with respect to addresses, the data verification component 118 may assign a high confidence (e.g., 0.3, 0.35, 0.38, etc.) to the address published by the mapping application. Based on the high confidence, the data verification component 118 may automatically update the address in the medical provider account 102.

In some examples, the data verification component 118 may automatically update the medical provider 106 specialty stored in the medical provider account 102 based on a determination that a threshold number and/or percentage of claims associated with services corresponding to a different specialty are received. In some examples, the threshold number of claims may be received over a threshold amount of time. For example, to update a medical specialty, the data verification component 118 may determine that the medical provider 106 has submitted the threshold number and/or percentage of claims for services associated with a different specialty (than that stored in the medical provider account 102) for at least three months.

In various examples, based on a determination that a probability of accuracy meets or exceeds a threshold probability, the data verification component 118 may automatically verify the medical provider data associated with the medical provider accounts 102, such as without human intervention. In such examples, the medical provider data stored in the medical provider accounts 102 may be automatically verified to ensure accurate information is available to members 108 and/or medical providers 106 accessing the medical provider data provided by the service provider computing device(s) 104.

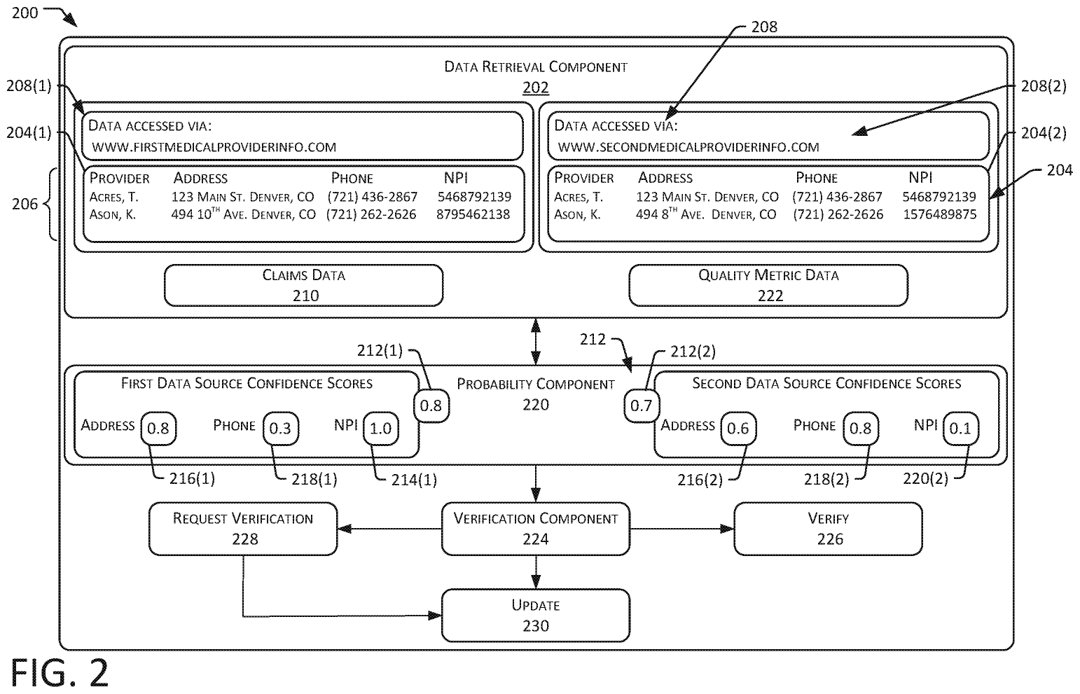

FIG. 2 is a block diagram 200 of example components associated with a data verification system. The data verification system may be performed by a data verification component, such as data verification component 118, of a service provider computing device, such as service provider computing device 104.

As illustrated the data verification system may include a data retrieval component 202. In various examples, the data retrieval component 202 may be configured to access one or more databases 204 including medical provider data 206. As illustrated in FIG. 2, the databases 204 may be accessed via one or more websites 208. In such examples, the medical provider data associated with the databases 204 may be stored on, published, or otherwise provided by a third-party computing device, such as third-party computing device 114. The website(s) 208 may include websites associated with governmental and/or regulatory organizations (e.g., CAQH, NCQA, NPPES, etc.), location related services (e.g., web-based mapping application, etc.), quality review services (e.g., crowd-sourced review forums, etc.), a business directory, and the like.

In the illustrative example, the medical provider data 206 may includes a provider name, address, phone number, and NPI. In other examples, the medical provider data 206 may include additional or alternative medical provider data, such as credentials, a type of medical practice affiliation (e.g., solo practice, group practice, etc.), alternative contact information (e.g., website, electronic mail, etc.), clinical visit times (e.g., average time, preferred (target) time, longest visit, shortest visit, etc.), insurance billing history, procedural history, procedural and/or practice specialties, provider quality metric (e.g., based on quality of service (e.g., based on feedback from members, professional organizations, awards earned, crowd-sourced ratings, etc.), ease of scheduling and/or delays associated with scheduling, and/or other information associated with the medical provider. Additionally, in the illustrative example, the databases 204(1) and 204(2) include the same types medical provider data 206 (e.g., both include provider name, address, phone number, and NPI). In other examples, the databases 204(1) and 204(2) may include the same and/or different types of medical provider data 206.

Additionally, in the illustrative example, the databases 204(1) and 204(2) include data associated with the same providers (e.g., Acres, T and Ason, K.). In some examples, the first database 204(1) may include the same and/or different providers from the second database 204(2). In such examples, the system may be configured to search across multiple websites 208 to access data associated with different medical providers, such as to verify medical provider data associated therewith stored in a medical provider database. For example, a medical provider database may include first medical provider data associated with a first medical provider and second medical provider data associated with a second medical provider. The data retrieval component 202 may determine that the first database 204(1) includes data associated with the first medical provider but not the second medical provider and the second database 204(2) includes data associated with the second medical provider but not the first medical provider. The data retrieval component 202 may thus access the first database 204(1) for verification of medical provider data associated with the first medical provider and the second database 204(2) for verification of medical provider data associated with the second medical provider.

In various examples, the data retrieval component 202 may be configured to pull the medical provider data 206 from the respective websites 208 for medical provider data verification. In some examples, the data retrieval component 202 may receive the medical provider data 206 from the respective websites 208, such as based on a request for the data. In some examples, the data retrieval component 202 may access particular websites 208, such as websites 208(1) and 208(2), based on a determination that a confidence score 212 associated therewith is above a threshold confidence score (e.g., 60%, 65%, 80%, etc.). In such examples, the data retrieval component 202 may limit access to websites 208 associated with accurate information.

In some examples, the data retrieval component 202 may access a first website 208(1) for data corresponding to a first type of medical provider data (e.g., first data set) and a second website 208(2) for data corresponding to a second type of medical provider data (e.g., second data set). In such examples, the determination to access the particular websites 208(1) or 208(2) for particular types of data may be based on confidence scores associated with the types of medical provider data published by the websites 208(1) and 208(2). For example, the data retrieval component 202 may access the first website 208(1) for NPI data based on a determination that an NPI confidence 214(1) is above a first threshold confidence and address data based on a determination that address confidence 216(1) is above a second threshold confidence. The data retrieval component 202 may access the second website 208(2) for telephone data based on a determination that a telephone number confidence 218(2) is above a third threshold confidence. In various examples, the data retrieval component 202 may receive the overall confidences 212 and/or the confidences in the type of medical data, such as address confidences 216(1) and 216(2), telephone number confidences 218(1) and 218(2), and/or NPI confidences 214(1) and 214(2) from a probability component 220. Though listed as NPI confidences 214(1) and 214(2), address confidences 216(1) and 216(2), and telephone number confidences 218(1) and 218(2), these are merely examples, and the probability component 220 may store confidences associated with one or more types of medical provider data provided by the websites 208.

Additionally or alternatively, the data retrieval component 202 may be configured to access claims data 210 associated with the medical providers. The claims data 210 may include data associated with insurance claims submitted to the service provider based on services rendered to members. In various examples, the claims data 210 may expressly include medical provider data, such as medical provider data submitted with the claim. For example, a claim may include an address and phone number associated with the medical provider submitting the claim.

In some examples, the claims data 210 may impliedly include medical provider data, such as a specialty associated with the medical provider based on particular services rendered. In such examples, the data retrieval component 202 may be configured to determine the medical provider specialty based on the claims data 210, such as based on a threshold number and/or percentage of services rendered related to the specialty. For example, based on a determination that 36% of the claims submitted by a medical provider, and represented in the claims data 210, correspond to ear, nose, and throat (ENT) services, the data retrieval component 202 may determine that the medical provider specialty is ENT.

In some examples, the data retrieval component 202 may determine a quality metric associated with medical providers based on quality metric data 222. The quality metric may represent a quality of service (e.g., based on feedback from members, professional organizations, awards earned, etc.), a quality of claims submissions (e.g., submitted on time with limited errors, etc.), and the like. In various examples, the quality metric data 222 may include data accessed via a crowd-sourced ranking platform, such as an application and/or website 208 configured to provide crowd-sourced rankings (e.g., rankings and/or scores based on data accumulated by the application and/or website 208). In such examples, the data retrieval component 202 may access the application or website 208 associated with the crowd-sourced ranking platform to determine the quality metric data 222. In some examples, the quality metric data 222 may include data received from one or more members, such as survey results or other feedback provided by members. In such examples, the data retrieval component 202 may determine the quality metric based on member input.

In some examples, the quality metric data 222 may be derived from the claims data 210. In such examples, the data retrieval component 202 may determine the quality metric based on one or more quality characteristics associated with the claims. The quality characteristic(s) may include timeliness of claim submissions, accuracy of claim submissions (e.g., limited errors, etc.), completeness of claim submissions, and the like. In various examples, the data verification system may be configured to verify a stored quality metric associated with a medical provider. In such examples, the service provider may ensure that accurate quality information is provided to members, such as for medical provider selection.

In various examples, the data verification system may include a probability component 220 configured to determine a probability of accuracy associated with medical provider information. The probability of accuracy may be determined based on data provided by the data retrieval component 202. As discussed above, the probability component 220 may determine confidence scores associated with websites 208. In some examples, the confidence scores may include an overall confidence that data published by a website 208 is accurate and/or confidences associated with particular types of data provided by the website 208, such as NPI confidence 214(1) and 214(2), address confidences 216(1) and 216(2), and telephone number confidences 218(1) and 218(2).

In various examples, the probability component may compare the data received from the data retrieval component 202 to medical provider data stored in a provider database, such as in medical provider accounts 102 of FIG. 1. In various examples, the probability component may determine a probability of accuracy of the medical provider data stored in the provider database based on the comparison. In some examples, based on a determination that the data in the provider database matches medical provider data 206 published by one or more data sources (e.g., via one or more websites 208), the probability component 220 may determine that the probability of accuracy associated with the data is high. For example, an address associated with a medical provider published on the first website 208(1) and on a second website 208(2) may match the address associated with the medical provider stored in the provider database. Accordingly, the probability component may determine the probability of accuracy to be 38%.

In some examples, the probability component 220 may determine the probability of accuracy based on the comparison and the confidence scores 212(1), 212(2), 214(1), 214(2), 216(1), 216(2), 218(1), and 218(2). In various examples, the probability of accuracy may include an aggregated probability based on data accessed via multiple data sources (one or more websites 208) and the confidences associated therewith. For example, an NPI associated with a medical provider published on the first website 208(1) may match an NPI stored in a provider database, but may differ from an NPI associated with the medical provider published on a second website 208(2). The probability component 220 may determine that the first NPI confidence score 214(1) associated with the first website 208(1) is 1.0 and a second NPI confidence score 214(2) associated with the second website 208(2) is 0.1. Accordingly, the probability component 220 may determine that the probability of accuracy associated with the NPI stored in the provider database is 32%.

In some examples, the probability component 220 may determine the probability of accuracy of medical provider data stored in a provider database based on claims data 210. As discussed above, the claims data 210 may include data corresponding to claims submitted to the service provider for payment for services rendered. The claims data 210 may include medical provider data, such as a name, address, phone number, electronic mail address, and/or other contact information. In some examples, the probability component 220 may compare the medical provider data provided in the claims data 210 to the medical provider data stored in the provider database to determine the probability of accuracy. For example, based on a match of the medical provider data (e.g., contact information) provided in the claims to the contact information stored in the provider database, the probability component may determine that the contact information associated with the medical provider in the provider database has a 100% probability of accuracy.

In some examples, the probability component 220 may determine a probability of accuracy of a specialty associated with a medical provider stored in the medical database based on the claims data 210. As discussed above, the data retrieval component 202 may be configured to determine the medical provider specialty based on the claims data 210, such as based on a threshold number and/or percentage of services rendered related to the specialty. The probability component 220 may receive the specialty data from the data retrieval component 202, such as with the claims data 210 and may compare the specialty data to a specialty associated with the medical provider stored in the database. Based on a comparison of the specialty data derived from the claims data 210 and the specialty data stored in the provider database, the probability component may determine a probability of accuracy of the specialty data stored in the provider database. For example, the probability component 220 may determine that 35% of the claims submitted by a medical provider are associated with a first specialty and the provider database associates the first specialty with the medical provider. Accordingly, the probability component 220 may determine that the first specialty stored in the provider database includes a 34% probability of accuracy. For another example, the probability component 220 may determine that 85% of the claims submitted by a medical provider are associated with a first specialty and the provider database associates a second specialty with the medical provider. Accordingly, the probability component 220 may determine that the second specialty stored in the provider database includes a 68% probability of accuracy.

In some examples, the probability component 220 may determine a probability of accuracy associated with a quality metric corresponding to a medical provider stored in the provider database and associated with the provider. In various examples, the probability component 220 may receive a current quality metric based on quality metric data 222 from the data retrieval component 202. In some examples, the probability component 220 may compare the current quality metric associated with a medical provider to a quality metric associated with the medical provider stored in the provider database. In such examples, the probability of accuracy may be based on the comparison of the current quality metric to the stored quality metric. In various examples, the probability component 220 may determine that a quality metric associated with a particular medical provider is verified based on a determination that no quality metric data 222 associated with the medical provider is received. For example, if quality metric data 222 includes a quality metric associated with a first medical provider but not a second medical provider, the probability component 220 may determine that the quality metric associated with the second medical provider is verified (e.g., probability of accuracy 100%) and may determine a probability of accuracy associated with the quality metric corresponding to the first medical provider.

In various examples, the probability component 220 may provide probabilities of accuracy associated with each type of medical provider data (e.g., address, telephone number, electronic mail address, NPI, provider specialty, etc.) stored in the provider database to a verification component 224, such as data verification component 118, for medical provider data verification. In such examples, the probabilities of accuracy may include aggregated probabilities based on a comparison of data against data published by one or more websites, claims submitted (e.g., claims data 210) by the medical providers, quality metric data 222, and the like.

In various examples, the verification component 224 may determine whether the probability of accuracy associated with each type of provider data for each medical provider meets or exceeds a threshold probability. Based on a determination that the probability of accuracy meets or exceeds the threshold probability, the verification component 224 may automatically verify the medical provider data at block 226, such as without human intervention. In such examples, the medical provider data stored in the medical provider database may be automatically verified to ensure accurate information is available to members and/or medical providers accessing the medical provider data provided by the service provider.

In some examples, based on a determination that the probability of accuracy is at or below the threshold probability, the verification component 224 may determine to request a manual verification of the provider data associated with the medical provider database at block 228. In some examples, the verification component 224 may provide an indication that verification is requested. In such examples, the indication may include the particular type of data to be verified (e.g., address, phone number, website, NPI, specialty, quality metric, etc.), a medical provider associated with the data (e.g., name, identifier, etc.), information regarding the third-party computing device corresponding to the disparate medical provider data (e.g., name of the data source, associated website, confidence scores, etc.), a date and/or time the medical provider data was last verified, and/or any other information to assist in data verification.

In various examples, based on the indication that verification is requested at block 228 (e.g., a verification request associated with medical provider data), a human may intervene to verify the medical provider data associated with the medical provider database. The verification may include a manual verification of to the medical provider data. In various examples, based on a determination that the stored medical provider data is inaccurate, the verifier may update the medical provider data at block 230. In some examples, the update may be based on the data received from the data retrieval component 202 and/or probability data determined by the probability component 220. In some examples, the update may include a date/time associated therewith, data associated with one or more resources used to verify and/or update the data, and/or any other information relevant to the verification and/or update of the data.

In some examples, based on a determination that the probability of accuracy is at or below the threshold probability and that confidence scores associated with the data sources (e.g., websites 208 and/or data sets associated with the databases 204) are above a threshold confidence score, the verification component 224 may automatically update the provider data associated with the medical provider database at block 230, such as without human intervention. In such examples, the medical provider database may be automatically updated to ensure current information is available to members and/or medical providers accessing the medical provider data. For example, a probability component 220 may receive an address corresponding to a medical provider from a mapping application with a confidence score of 38%. The probability component may determine that the address associated with the medical provider stored in a medical provider database does not match the address published by the mapping application and that a corresponding probability of accuracy of the stored address is 65%. Based on a determination that the probability of accuracy is below a threshold probability and that the confidence score associated with the mapping application is above a threshold confidence score, the verification component 224 may determine to automatically update the address in the medical provider database at block 230.

In some examples, the verification component 224 may cause a medical provider specialty stored in the medical provider database to be automatically update at block 230 based on a determination that a threshold number and/or percentage of claims associated with services corresponding to a different specialty are received. In some examples, the threshold number of claims may be received over a threshold amount of time. For example, to update a medical specialty, the verification component 224 may determine that the medical provider has submitted the threshold number and/or percentage of claims for services associated with a different specialty (than that stored in a medical provider account) for at least three months.

In some examples, the verification component 224 may cause a quality metric associated with a medical provider stored in the medical provider database to be automatically update at block 230 based on a determination that the probability of accuracy associated therewith is below a threshold. In some examples, the automatic verification may be based on a data source associated with quality metric data 222 resulting in the probability determination. In some examples, the automatic verification may be based on a threshold number of reviews and/or member submitted surveys, a confidence score associated with a crowd-sourced review website 208, a timeliness score and/or an accuracy score associated with submitted claims (e.g., average timeliness, average accuracy score, etc.), and the like.

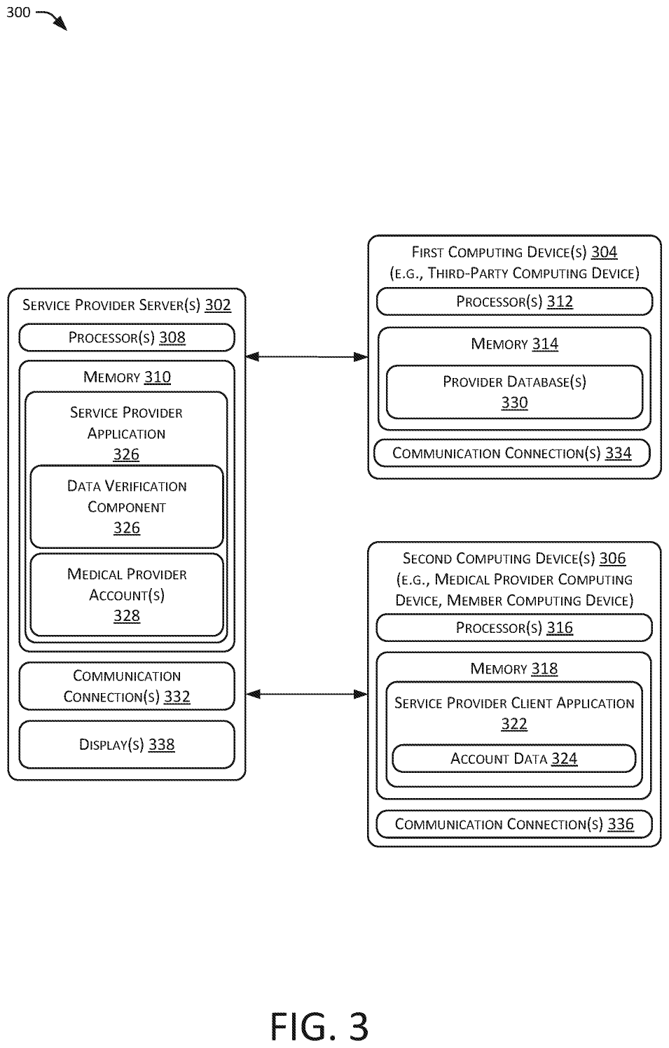

FIG. 3 illustrates a block diagram illustrating an example system 300 of computing devices usable to implement example techniques described herein. For example, FIG. 3 illustrates example computing devices including service provider server(s) 302, one or more first computing devices 304, and one or more second computing devices 306, that interact over a network, such as network 116 in FIG. 1. By way of example and not limitation, the service provider server(s) 302 may be representative of servers used to implement the system 100, the first computing device(s) 304 may be representative of the third-party computing devices 114, and the second computing device(s) 306 may be representative of the medical provider computing device 112 associated with the medical provider 106 and/or member computing device 110 associated with the member 108.

The service provider server(s) 302 may comprise one or more individual servers or other computing devices that may be physically located in a single central location or may be distributed at multiple different locations. The service provider server(s) 302 may be hosted privately by an entity administering all or part of the communications network (e.g., a utility company, a governmental body, distributor, a retailer, manufacturer, etc.), or may be hosted in a cloud environment, or a combination of privately hosted and cloud hosted services.

Each of the computing devices described herein may include one or more processors and/or memory. Specifically, in the illustrated example, service provider server(s) 302 include one or more processors 308 and memory 310, first computing device(s) 304 includes one or more processors 312 and memory 314, and second computing device(s) 306 includes one or more processors 316 and memory 318. By way of example and not limitation, the processor(s) may comprise one or more Central Processing Units (CPUs), Graphics Processing Units (GPUs), or any other device or portion of a device that processes electronic data to transform that electronic data into other electronic data that may be stored in registers and/or memory. In some examples, integrated circuits (e.g., ASICs, etc.), gate arrays (e.g., FPGAs, etc.), and other hardware devices may also be considered processors in so far as they are configured to implement encoded instructions.

The memory may comprise one or more non-transitory computer-readable media and may store an operating system and one or more software applications, instructions, programs, and/or data to implement the methods described herein and the functions attributed to the various systems. In various implementations, the memory may be implemented using any suitable memory technology, such as static random-access memory (SRAM), synchronous dynamic RAM (SDRAM), nonvolatile/Flash-type memory, or any other type of memory capable of storing information. The architectures, systems, and individual elements described herein may include many other logical, programmatic, and physical components, of which those shown in the accompanying figures are merely examples that are related to the discussion herein.

As shown in FIG. 3, service provider server(s) 302 includes a service provider application 320 and second computing device(s) 306 includes service provider client application 322 that enables interaction of content between the service provider server(s) 302 and the second computing device(s) 306. For example, content (e.g., medical provider data, claims submissions, member data, member surveys and/or feedback regarding medical providers, etc.) may be shared among users associated with service provider accounts (e.g., member accounts, medical provider accounts, etc.) of an insurance provider network provided by the service provider system and may include sharing content in accordance with an account of a user that is restricted, such as based on health information privacy rules and/or regulations. In some examples, the service provider client application 322 may enable an interface associated with a second computing device 306 to access content, to view content, and to generate content, such as, for example, insurance claims, medical provider surveys, and the like. In particular examples, service provider server(s) 302 send instructions to present, transmit, and receive content including medical provider data, as discussed with reference to FIG. 2 and FIG. 3. In various examples, the service provider server(s) 302 send instructions to store data associated with a medical provider, such as in account data 324. For example, the second computing device 306 may include a medical provider computing device associated with a medical provider. Medical provider data associated with the medical provider and/or one or more other medical providers may be stored in the account data 324.

FIG. 3 further illustrates service provider server(s) 302 as including data verification component 326 and medical provider accounts 328 to enable content such as medical provider data to be shared among the computing devices. In various examples, the verification component 326 may be configured determine whether to automatically verify medical provider data or to request manual verification thereof. In various examples, the automatic verification may be determined based on a comparison of medical provider data stored in the medical provider account(s) 328 to data provided by one or more data sources. The data sources may include the first computing device(s) 304 and/or the second computing device(s) 306.

In various examples, the data verification component 326 may access provider databases 330 associated with one or more first computing device(s) 304. The provider databases 330 may include medical provider data. The provider databases 330 may include different types of medical provider data, based on a third-party service provider (e.g., data source) managing a first computing device 304. For example, a first computing device 304 associated with a mapping application may include a name, address, and phone number associated with a medical provider and another first computing device 304 associated with a crowd-sourced ranking system may include a name and a quality metric associated with the medical provider. In such an example, the provider databases 330 may include the medical provider data associated with the service and/or information provided by the data source. In various examples, the data verification component 326 may assign a confidence score to each provider database 330 (e.g., overall confidence score) and/or a confidence score to each data set (e.g., type of data) associated therewith. For example, a provider database 330 may have associated therewith a first confidence score associated with medical provider addresses and a second confidence score associated with medical provider phone numbers, and the like.

In some examples, the data verification component 326 may receive insurance claims from the one or more second computing device(s) 306. The insurance claims may include claims for payment for services rendered to one or more members. The insurance claims may include medical provider data, such as name, address, and/or phone number associated with the medical provider. In various examples, the data verification component 326 may be configured to derive medical provider data from the insurance claims. In such example, the data verification component 326 may receive the insurance claims and determine a specialty associated with based on services performed by the medical provider.

In some examples, the data verification component 326 may verify a quality metric associated with a medical provider based on the claims data. In such examples, the quality metric may be based on part on timeliness and/or accuracy of insurance claims submissions. In some examples, the quality metric may be based in part on member feedback regarding services rendered by and/or interactions with the medical provider. In such examples, the data verification component 326 may receive the member feedback from the one or more second computing device(s) 306. In some examples, the quality metric may be based in part on data received from a quality review website, such as a crowd-sourced review forum or other website including quality reviews of medical providers. In such examples, the data verification component 326 may access the respective websites to determine the data associated with the quality metric.

In various examples, the data verification component 326 may determine a probability of accuracy of medical provider data based in part on a comparison of the stored data, such as in a medical provider account 328, to the data accessed via one or more data sources (e.g., websites, claims data, member feedback, etc.). The probability of accuracy may represent a probability that the data is accurate at a time in which the verification is conducted. As discussed above, the probability of accuracy may be based on confidence scores associated with the data source.

In some examples, based on a determination that the probability of accuracy associated with a particular type of data corresponding to a particular medical provider is at or above (e.g., meets or exceeds) a threshold, the data verification component 326 may automatically verify the data. In some examples, based on a determination that the probability of accuracy associated with a particular type of data corresponding to a particular medical provider is at or below the threshold, the data verification component 326 may request human intervention for verification. In some examples, the data verification component 326 may provide an indication that manual verification is required. The indication may include the particular type of data to be verified (e.g., address, phone number, website, NPI, specialty, etc.), a medical provider associated with the data (e.g., name, identifier, etc.), information regarding the data source (e.g., name of the data source, confidence scores, etc.), a date and/or time the data was last verified, and/or any other information to assist in data verification.

In some examples, based on a determination that the probability of accuracy associated with a particular type of data corresponding to a particular medical provider is at or below the threshold, the data verification component 326 may automatically update the medical provider data stored in the medical provider account 328 and associated with the medical provider. As discussed above, the data verification component 326 may automatically update the medical provider data based on a determination that a confidence score associated with the data source is at or above a threshold confidence. In some examples, the data verification component 326 may automatically update the medical provider data based on a receipt of a threshold number and/or threshold percentage of claims received from the medical provider being associated with a particular specialty. In some examples, the threshold number and/or percentage of insurance claims may be received over a threshold period of time (e.g., threshold number and/or percentage of insurance claims received over a year, etc.).

As shown in FIG. 3, service provider server(s) 302 may include communications connection(s) 332, first computing device(s) 304 may include communications connection(s) 334, and second computing device(s) 306 may include communications connection(s) 336 that enable communication between at least the service provider server(s) 302 and one or more of the first computing device(s) 304, and the second computing device(s) 306.

The communication connection(s) 332, 334, and/or 336 may include physical and/or logical interfaces for connecting service provider server(s) 302, first computing device(s) 304, and/or second computing device(s) 306 to another computing device or a network, such as network(s) 116. For example, the communications connection(s) 332, 334, and/or 336 may enable Wi-Fi-based communication such as via frequencies defined by the IEEE 802.11 standards, short range wireless frequencies such as Bluetooth®, cellular communication (e.g., 2G, 2G, 4G, 4G LTE, 5G, etc.) or any suitable wired or wireless communications protocol that enables the respective computing device to interface with the other computing device(s).

In the illustrative example, the service provider server(s) 302 may include one or more displays 338 for presenting data. In some examples, the data may include instructions to verify medical provider data, notifications of automatic verifications and/or updates to medical provider data, notifications of receipt of insurance claims, member feedback, or the like. Depending on the type of computing device used as the service provider server(s) 302, the display 338 may employ any suitable display technology. For example, the display 338 may include a liquid crystal display, a plasma display, a light emitting diode display, an OLED (organic light-emitting diode) display, an electronic paper display, or any other suitable type of display able to present digital content thereon. In some examples, the display 338 may have a touch sensor associated with the display 338 to provide a touchscreen display configured to receive touch inputs for enabling interaction with a graphical user interface presented on the display 338, such as a graphical user interface associated with a manual verification of medical provider data and/or other services provided by the service provider. Accordingly, implementations herein are not limited to any particular display technology.

While FIG. 3 is provided as an example system 300 that can be used to implement techniques described herein, the techniques described and claimed are not limited to being performed by the system 300, nor is the system 300 limited to performing the techniques described herein.

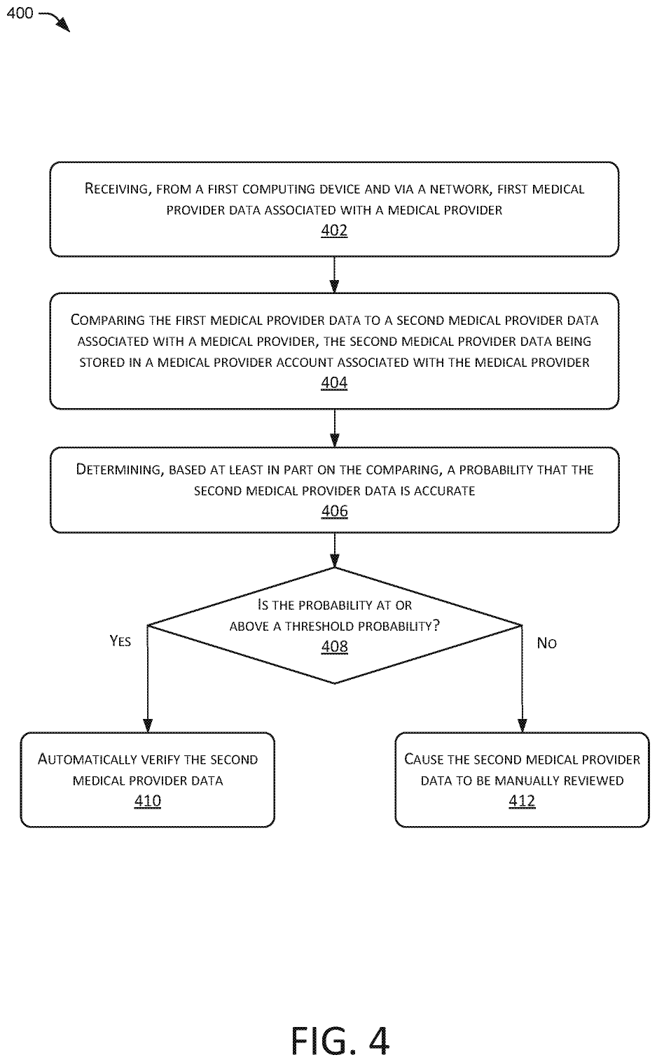

FIGS. 4-6 illustrate example processes in accordance with embodiments of the disclosure. These processes are illustrated as logical flow graphs, each operation of which represents a sequence of operations that may be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations may be combined in any order and/or in parallel to implement the processes.

FIG. 4 illustrates an example processes 400 for automatically verifying medical provider data, utilizing the techniques described herein. In some instances, some or all of process 400 may be performed by one or more components in the systems 100 or 300. By way of example and not limitation, the service provider computing device (e.g., service provider) referred to in process 400 may be representative of a computing device associated with the service provider 104 or service provider server(s) 302, the third-party computing device (e.g., third-party device) referred to in process 400 may be representative of the third-party computing device(s) 114 and/or first computing device(s) 304, and the member computing device and/or medical provider computing device referred to in process 400 may be representative of the member computing device(s) 110, the medical provider computing devices 112 and/or the second computing device(s) 306. However, the process 400 is not limited to being performed by the system 100 or 300.

At operation 402, the process may include receiving, from a computing device and via a network, first medical provider data associated with a medical provider. In various examples, the service provider computing device may receive the first medical provider data based on a request for medical provider data. In some examples, the service provider computing device may send the request for medical provider data based on a determination that a confidence associated with a data source associated with the computing device is above a threshold confidence. In some examples, the confidence may be associated with the data source overall and/or a type of provider data (e.g., data set) provided by the data source. For example, a data source may have a first confidence associated with an overall accuracy of data published, a second confidence associated with a first type of provider data, and a third confidence associated with a second type of provider data. The data source may be accessed and/or provider data requested therefrom based on the first, second, and/or third confidences.

At operation 404, the may include comparing the first medical provider data to a second medical provider data associated with a medical provider, the second medical provider data being stored in a medical provider account associated with the medical provider. In various examples, the service provider computing device may compare the stored medical provider data and medical provider data received from a plurality of data sources (e.g., a plurality of computing devices).

In some examples, the service provider computing device may determine that the first medical provider data received from a data source and the second medical provider data substantially match. In such examples, the substantial match may be based on a determination that a threshold number of numbers and/or letters of the first medical provider data are the same and/or in a same order as the numbers and/or letters of the second medical provider data. For example, the service provider computing device may determine that a street number and street name (e.g., address) received from a data source is the same as a street number and street name stored in a medical provider account.

In some examples, the medical provider data may substantially match based on a determination that a difference between the first medical provider data and the second medical provider data is less than a threshold difference (e.g., a number and/or percentage of letters and/or numbers, an order of the letters and/or numbers, etc.). For example, the service provider computing device may determine that the addresses differ by a single letter. The service provider computing device may determine that the difference is less than a threshold difference (e.g., may be attributable to a typo) and that the addresses substantially match one another. In some examples, the threshold difference may be based on the type of data compared (e.g., address, professional identifier (e.g., NPI), etc.). For example, a first threshold difference associated with a medical provider address may include a higher tolerance (e.g., 2 or 3 numbers and/or letters differ in value or placement) than a second threshold difference associated with a medical provider professional identifier (e.g., 0 numbers differ for a substantial match).

In various examples, the service provider computing device may determine a difference between the first medical provider data and the second medical provider data is above a threshold difference. In such examples, the service provider computing device may determine that the first medical provider data and the second medical provider data do not match.

At operation 406, the process may include determining, based at least in part on the comparing, a probability that the second medical provider data is accurate. The probability may be based at least in part on the difference between the first medical provider data and the second medical provider data. In some examples, the probability may be based on the confidence associated with the data source. In such examples, the probability may include a weighted probability based on the confidence. In some examples, the probability may include an aggregated probability that the stored medical provider data is accurate, based on data received from a plurality of data sources. In some examples, the aggregated probability may include a weighted probability based on the confidence associated with each data source of the plurality of data sources.

At operation 408, the process may include determining whether the probability is at or above a threshold probability (e.g., 75%, 80%, 95%, etc.). In some examples, the threshold probability may be based on a type of data. In such examples, a first type of data may have a different threshold probability than a second type of data. In some examples, the service provider computing device may associate a higher threshold with a certain type of data, such as based on an importance of accuracy associated therewith. For example, the service provider computing device may determine that medical provider data associated with a phone number to reach a medical provider is more important to members than a website associated therewith. The service provider computing device may thus assign a threshold of 95% to data associated with a phone number and a threshold of 80% to data associated with a website.

Based on a determination that the probability is at or above the threshold probability (“Yes” at operation 408), the process may include, at operation 410, automatically verifying the second medical provider data. In various examples, the automatic verification may include a verification without human intervention. In some examples, the automatic verification may include storing metadata associated with the verification, such as a date and/or time associated with the verification, an indication of the data source associated with the first medical provider data used to verify the second medical provider data, and the like.

Based on a determination that the probability is below the threshold probability (“No” at operation 408), the process may include, at operation 412, causing the second medical provider data to be manually reviewed. In some examples, the service provider computing device may surface an instruction to manually verify the medical provider data. In some examples, the instruction may include the particular type of data to be verified (e.g., address, phone number, website, NPI, specialty, etc.), a medical provider associated with the data (e.g., name, identifier, etc.), information regarding the data source (e.g., name of the data source, confidence scores, etc.), a date and/or time the data was last verified, and/or any other information to assist in data verification.

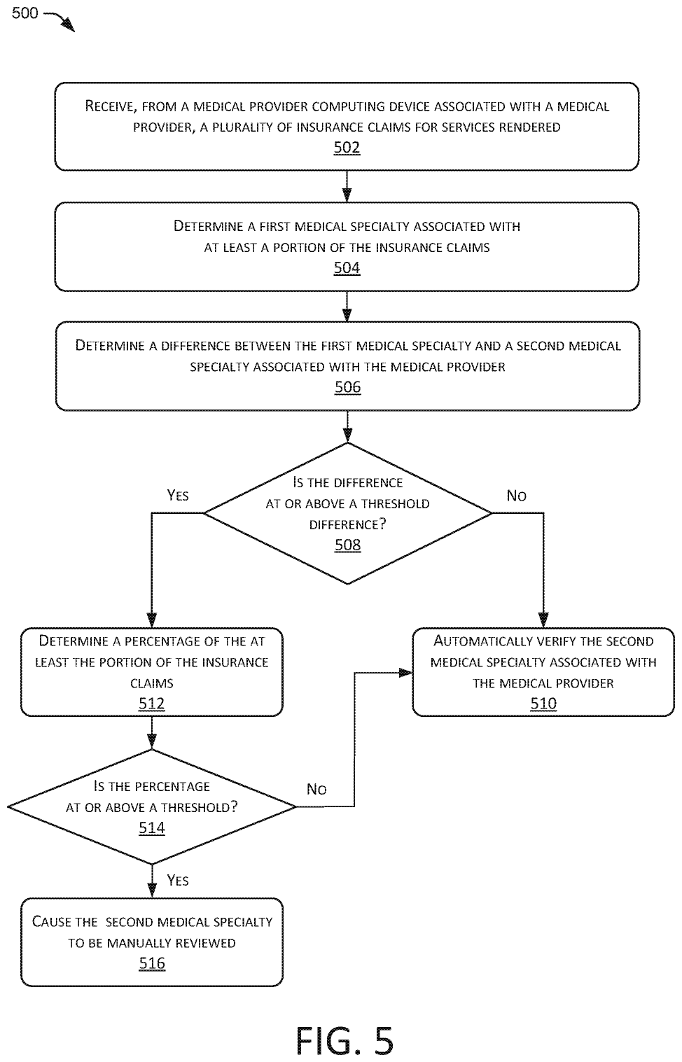

FIG. 5 illustrates an example processes 500 for automatically verifying a medical provider specialty based on received insurance claims, utilizing the techniques described herein. In some instances, some or all of process 400 may be performed by one or more components in the systems 100 or 300. By way of example and not limitation, the service provider computing device (e.g., service provider) referred to in process 500 may be representative of a computing device associated with the service provider 104 or service provider server(s) 302, the third-party computing device (e.g., third-party device) referred to in process 500 may be representative of the third-party computing device(s) 114 and/or first computing device(s) 304, and the member computing device and/or medical provider computing device referred to in process 500 may be representative of the member computing device(s) 110, the medical provider computing devices 112 and/or the second computing device(s) 306. However, the process 500 is not limited to being performed by the system 100 or 300.

At operation 502, the process may include receiving, from a medical provider computing device associated with a medical provider, a plurality of insurance claims for services rendered. In various examples, the service provider computing device may receive the plurality of insurance claims based on a service provided thereby, such as a medical insurance service. The insurance claims may include requests for payments for the services rendered to members associated with the service provider (e.g., members of the medical insurance service).

At operation 504, the process may include determining a first medical specialty associated with at least a portion of the insurance claims. In various examples, the service provider computing device may be configured to determine a medical specialty associated with each of the services rendered. For example, an insurance claim for an annual check-up may be associated with a general practice physician and an insurance claim for an angioplasty may be associated with a cardiologist.

At operation 506, the process may include determining a difference between the first medical specialty and a second medical specialty associated with the medical provider. The second medical specialty may be associated with the medical provider, such as in a medical provider account stored on a data store. In some examples, the second medical specialty may be provided by the medical provider, such as in establishing an account with the service provider. In various examples, the second medical specialty may be determined by the service provider computing device based on previous insurance claims submitted by the medical provider.

At operation 508, the process may include determining whether the difference between the first medical specialty and the second medical specialty is at or above a threshold difference. In some examples, the threshold difference may be based on types of services rendered, related specialties, and the like. For example, a second (stored in a medical provider account) medical specialty associated with a medical provider may include cardiology. The service provider computing device may receive a plurality of insurance claims from the medical provider related to general practice services rendered. The service provider computing device may determine that the difference between cardiology (e.g., a specialty that focuses on a particular organ and/or system) and general practice (e.g., general services related to preventative care and health education) is above the threshold difference. For another example, a portion of the plurality of insurance claims from the medical provider with the associated medical specialty of cardiology may include tests for kidney failure. Based at least in part on a determination that kidney failure may be related to heart failure, the service provider computing device may determine that the services associated with kidney failure tests are sufficiently related to cardiology services and that the difference between the two is below the threshold difference.

Based on a determination that the difference is below the threshold difference (“No” at operation 508), the process may include, at operation 510, automatically verifying the second medical specialty associated with the medical provider. In various examples, the automatic verification may include a verification without human intervention. In some examples, the automatic verification may include storing metadata associated with the verification, such as a date and/or time associated with the verification, an indication of the at least the portion of the insurance claims used to verify the medical specialty, and the like.

Based on a determination that the difference is at or above the threshold difference (“Yes” at operation 508), the process may include, at operation 512, determining a percentage of the at least the portion of the insurance claims. The percentage may include a ratio of the number of insurance claims associated with the first medical specialty to the total number of insurance claims submitted by the medical provider. In some examples, the percentage may be associated with the total number of insurance claims submitted over a pre-determined period of time (e.g., a day, two weeks, a month, three months, a year, etc.).

At operation 514, the process may include determining whether the percentage is above a threshold percentage. In some examples, the threshold percentage may represent a minimum percentage of claims for services associated with a specialty to be rendered in order to indicate a medical specialty and/or focus of a medical practice.

Based on a determination that the percentage is below the threshold percentage (“No” at operation 514), the process may include automatically verifying the second medical specialty associated with the medical provider, such as that described with regard to operation 510.

Based on a determination that the percentage is at or above the threshold percentage (“Yes” at operation 514), the process, at operation 516, may include causing the second medical specialty to be manually reviewed. In some examples, the service provider computing device may surface an instruction to manually verify the second medical provider specialty. In some examples, the instruction may include the medical provider associated with the data (e.g., name, identifier, etc.), information associated with the insurance claims, a date and/or time the second medical specialty was last verified, and/or any other information to assist in medical specialty verification.

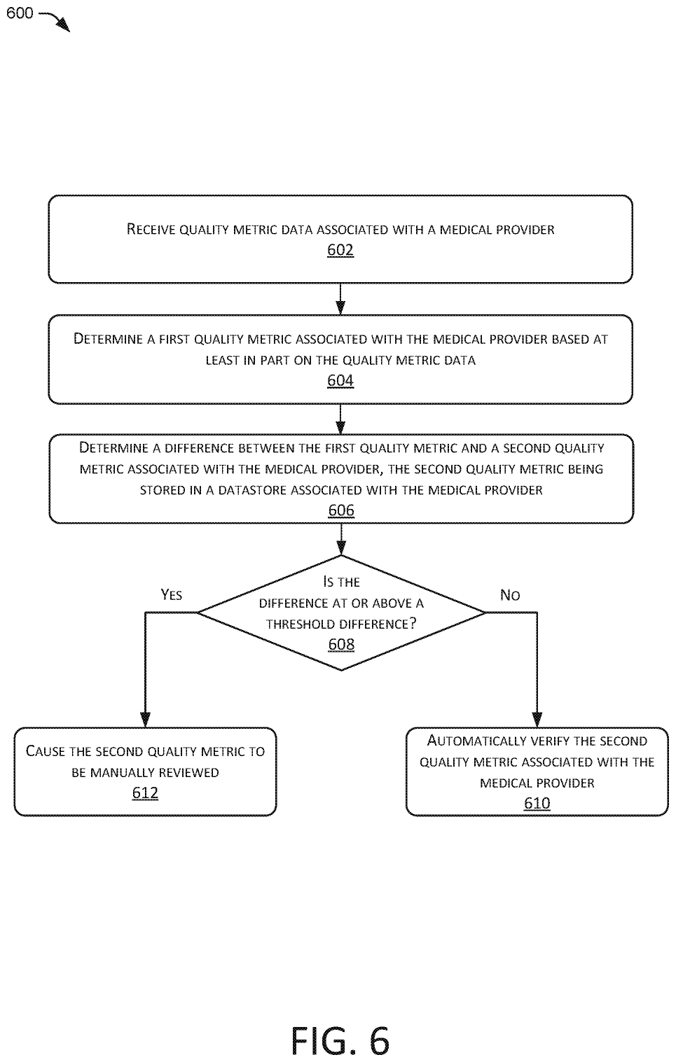

FIG. 6 illustrates an example processes 600 for automatically verifying a medical provider quality metric, utilizing the techniques described herein. In some instances, some or all of process 600 may be performed by one or more components in the systems 100 or 300. By way of example and not limitation, the service provider computing device (e.g., service provider) referred to in process 600 may be representative of a computing device associated with the service provider 104 or service provider server(s) 302, the third-party computing device (e.g., third-party device) referred to in process 600 may be representative of the third-party computing device(s) 114 and/or first computing device(s) 304, and the member computing device and/or medical provider computing device referred to in process 600 may be representative of the member computing device(s) 110, the medical provider computing devices 112 and/or the second computing device(s) 306. However, the process 600 is not limited to being performed by the system 100 or 300.

At operation 602, the process may include receiving quality metric data associated with a medical provider. The quality metric data may represent data associated with a quality of service (e.g., based on feedback from members, professional organizations, awards earned, etc.), a quality of claims submissions (e.g., submitted on time with limited errors, etc.), and the like. In various examples, the quality metric data may include quality review data accessed via a crowd-sourced ranking platform (e.g., crowd-sourced review source), such as an application or website configured to provide crowd-sourced rankings. In such examples, the crowd-sourced ranking platform may receive the quality review data and accumulate the data into quality metric data to provide to the service provider computing device and/or other individuals (e.g., patients, medical providers, etc.) or entities with access to the crowd-sourced ranking platform. In some examples, the quality metric data may include feedback data received from one or more members, such as survey results or other feedback provided by members. In some examples, the quality metric data may be determined based on insurance claim submissions. In such examples, the service provider computing device may determine the quality metric data based on one or more quality metrics associated with insurance claims, such as timeliness of submissions, accuracy of submissions, and the like.

At operation 604, the process may include determining a first quality metric associated with the medical provider based at least in part on the quality metric data. In some examples, the quality metric may represent the quality of service, quality of claim submissions, adherence to insurance claim guidance (e.g., timelines, accurate submissions, etc.), and the like.

At operation 606, the process may include determining a difference between the first quality metric and a second quality metric associated with the medical provider, the second quality metric being stored in a database associated with the medical provider. For example, a medical provider may have associated therewith in a medical provider account stored on the database, a quality metric of 4.8 out of 5.0 stars. The service provider computing device may receive quality metric data and may determine an updated quality metric of 4.9 out of 5.0 stars. The service provider computing device may determine that the difference is 0.1 star. In some examples, the service provider computing device may provide the second quality metric and/or quality metric data associated therewith to members, such as to inform a decision regarding a medical provider to choose for future services.

At operation 608, the process may include determining whether the difference is at or above a threshold difference. The threshold difference may represent a numerical or other rating and/or ranking difference between the first quality metric and the second quality metric. For example, the threshold difference may include a 0.3-point difference in a rating, a 10% decrease in a rating, or the like. Though these numbers are merely provided for illustrative purposes and are not intended to be so limiting.

Based on a determination that the difference is below the threshold difference (“No” at operation 608), the process may include, at operation 610, automatically verifying the second quality metric associated with the medical provider. In various examples, the automatic verification may include a verification without human intervention. In some examples, the automatic verification may include storing metadata associated with the verification, such as a date and/or time associated with the verification, an indication of the data source(s) associated with the quality metric data used to verify the second quality metric, and the like.

Based on a determination that the difference is at or above the threshold difference (“Yes” at operation 608), the process may include, at operation 612, causing the second quality metric to be manually reviewed. In some examples, the service provider computing device may surface an instruction to manually verify the second quality metric. In some examples, the instruction may include the particular type of data to be verified (e.g., quality metric, etc.), a medical provider associated with the quality metric (e.g., name, identifier, etc.), information regarding the data source (e.g., name of the data source, confidence scores, etc.), a date and/or time the data was last verified, and/or any other information to assist in data verification.

As stated above, the order in which the operations are described is not intended to be construed as a limitation, and any number of the described operations may be combined in any order and/or in parallel to implement the processes. In some embodiments, one or more operations of the above-described methods may be omitted entirely. By way of example and not limitation, operations 502-510 may be performed without operations 512-516. Moreover, the methods described herein can be combined in whole or in part with each other or with other methods.

The various techniques described herein may be implemented in the context of computer-executable instructions or software, such as program modules, that are stored in computer-readable storage and executed by the processor(s) of one or more computing devices such as those illustrated in the figures. Generally, program modules include routines, programs, objects, components, data structures, etc., and define operating logic for performing particular tasks or implement particular abstract data types.

Other architectures may be used to implement the described functionality and are intended to be within the scope of this disclosure. Furthermore, although specific distributions of responsibilities are defined above for purposes of discussion, the various functions and responsibilities might be distributed and divided in different ways, depending on circumstances.

Similarly, software may be stored and distributed in various ways and using different means, and the particular software storage and execution configurations described above may be varied in many different ways. Thus, software implementing the techniques described above may be distributed on various types of computer-readable media, not limited to the forms of memory that are specifically described.

### CONCLUSION

Although the discussion above sets forth example implementations of the described techniques, other architectures may be used to implement the described functionality, and are intended to be within the scope of this disclosure. Furthermore, although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A method comprising:
receiving, from a computing device and via a network, first medical provider data associated with a medical provider;
comparing the first medical provider data to a second medical provider data associated with the medical provider, the second medical provider data being stored in a medical provider account associated with the medical provider;
determining, based at least in part on the comparing, a probability that the second medical provider data is accurate; and
performing at least one of:
based at least in part on a determination that the probability is at or above a threshold probability, automatically verifying accuracy of the second medical provider data; or
based at least in part on a determination that the probability is below the threshold probability, causing the second medical provider data to be manually verified.

2. The method of claim 1, further comprising:
determining that a confidence associated with a data source corresponding to the computing device is above a threshold confidence; and
sending a request to the computing device for the first medical provider data based at least in part on the confidence being above the threshold confidence,
wherein receiving the first medical provider data is based at least in part on the request.

3. The method of claim 2, wherein the probability that the second medical provider data is accurate is based at least in part on the confidence.

4. The method of claim 1, wherein the first medical provider data and the second medical provider data comprise at least one of:
one or more names associated with the medical provider;
an address associated with the medical provider;
a phone number associated with the medical provider;
a website associated with the medical provider;
an electronic mail address associated with the medical provider;
a medical specialty associated with the medical provider;
a certification associated with the medical provider; or
a quality metric associated with the medical provider.

5. The method of claim 1, wherein the computing device is a first computing device, the method further comprising:
receiving, from a second computing device and via the network, third medical provider data associated with the medical provider;
determining that a confidence associated with the third medical provider data is above a threshold confidence;
determining a difference between the third medical provider data and the second medical provider data stored in the medical provider account;
determining that the difference meets or exceeds a threshold difference; and
automatically updating the medical provider account to include the third medical provider data based at least in part on the confidence and the difference.

6. The method of claim 1, wherein the computing device is a first computing device, the method further comprising:
receiving, from a second computing device, quality metric data associated with the medical provider;
determining a first quality metric associated with the medical provider based at least in part on the quality metric data;
determining a difference between the first quality metric and a second quality metric stored in the medical provider account associated with the medical provider; and
performing at least one of:
based at least in part on a determination that the difference is less than a threshold difference, automatically verifying the second quality metric associated with the medical provider; or
based at least in part on a determination that the difference is at or above a threshold difference, causing the second quality metric to be manually verified.

7. The method of claim 6, wherein the quality metric data comprises at least one of:
quality review data associated with services rendered by the medical provider, wherein the quality review data is accumulated by a crowd-sourced review service;
a timeliness metric associated with insurance claims submitted by the medical provider;
an accuracy metric associated with the insurance claims; or
feedback data generated by a patient associated with a service rendered by the medical provider.

8. A computing system comprising:
one or more processors; and
computer readable media storing instructions that, when executed by the one or more processors, cause the computing system to:
receive, from a computing device and via a network, first medical provider data associated with a medical provider;
compare the first medical provider data to a second medical provider data associated with the medical provider, the second medical provider data being stored in a medical provider account associated with the medical provider;
determine, based at least in part on the comparing, a probability that the second medical provider data is accurate; and
based at least in part on a determination that the probability is at or above a threshold probability, automatically verify accuracy of the second medical provider data.

9. The computing system of claim 8, the instructions further causing the computing system to:
determine that a confidence associated with a data source corresponding to the computing device is above a threshold confidence; and
send a request to the computing device for the first medical provider data based at least in part on the confidence being above the threshold confidence,
wherein receiving the first medical provider data is based at least in part on the request.

10. The computing system of claim 8, the instructions further causing the computing system to:
determine that a confidence associated with a data source corresponding to the computing device is above a threshold confidence,
wherein the probability that the second medical provider data is accurate is based at least in part on the confidence associated with the data source.

11. The computing system of claim 8, wherein the computing device is a first computing device, the instructions further causing the computing system to:
receive, from a second computing device and via the network, third medical provider data associated with the medical provider;
determine that a confidence associated with the third medical provider data is above a threshold confidence;
determine a difference between the third medical provider data and the second medical provider data stored in the medical provider account;
determine that the difference meets or exceeds a threshold difference; and
automatically update the medical provider account to include the third medical provider data based at least in part on the confidence and the difference.

12. The computing system of claim 8, wherein the computing device is a first computing device, the instructions further causing the computing system to:
receive, from a second computing device associated with a medical provider, a plurality of claims for services rendered;
determine a first medical specialty associated with the services rendered;
determine a difference between the first medical specialty and a second medical specialty stored in the medical provider account;
determine that the difference is less than a threshold difference; and
automatically verifying the second medical specialty associated with the medical provider.

13. The computing system of claim 8, wherein the computing device is a first computing device, the instructions further causing the computing system to:
receive, from a second computing device associated with a medical provider, a plurality of claims for services rendered;
determine that a first medical specialty associated with a first set of claims of the plurality of claims differs from a second medical specialty stored in the medical provider account;
determine that a percentage of claims associated with the first set of claims is above a threshold percentage; and
performing at least one of:
automatically storing the first medical specialty associated with the medical provider in the medical provider account; or
causing a manual verification of the second medical specialty to be performed.

14. The computing system of claim 8, wherein the first medical provider data and the second medical provider data comprise at least one of:
one or more names associated with the medical provider;
an address associated with the medical provider;
a phone number associated with the medical provider;
a website associated with the medical provider;
an electronic mail address associated with the medical provider;
a medical specialty associated with the medical provider;
a certification associated with the medical provider; or
a quality metric associated with the medical provider.

15. One or more computer readable media storing instructions that, when executed by one or more processors of a computing device, cause the computing device to:
receive, from a remote computing device and via a network, first medical provider data associated with a medical provider;
compare the first medical provider data to a second medical provider data associated with the medical provider, the second medical provider data being stored in a medical provider account associated with the medical provider;
determine, based at least in part on the comparing, a probability that the second medical provider data is accurate; and
based at least in part on a determination that the probability is at or above a threshold probability, automatically verify accuracy of the second medical provider data.

16. The one or more computer readable media of claim 15, the instructions further causing the computing device to:
determine that a confidence associated with a data source corresponding to the remote computing device is above a threshold confidence; and
send a request to the remote computing device for the first medical provider data based at least in part on the confidence being above the threshold confidence,
wherein receiving the first medical provider data is based at least in part on the request and
wherein the probability is based at least in part on the confidence.

17. The one or more computer readable media of claim 15, wherein the probability is a first probability, the instructions further causing the computing device to:
receive, from the remote computing device and via the network, third medical provider data associated with the medical provider;
compare the third medical provider data to a fourth medical provider data associated with the medical provider, the fourth medical provider data being stored in the medical provider account associated with the medical provider;
determine, based at least in part on the comparing, a second probability that the fourth medical provider data is accurate; and
based at least in part on a determination that the second probability is below the threshold probability, surface an instruction to manually verify the fourth medical provider data.

18. The one or more computer readable media of claim 15, wherein the remote computing device is a first remote computing device, the instructions further causing the computing device to:
receive, from a second remote computing device associated with a medical provider, a plurality of claims for services rendered;
determine that a first medical specialty associated with a first set of claims of the plurality of claims differs from a second medical specialty stored in the medical provider account;
determine that a percentage of claims associated with the first set of claims is above a threshold percentage; and
performing at least one of:
automatically storing the first medical specialty associated with the medical provider in the medical provider account; or
causing a manual verification of the second medical specialty to be performed.

19. The one or more computer readable media of claim 15, wherein the remote computing device is a first remote computing device, the instructions further causing the computing device to
receive, from a second remote computing device, quality metric data associated with the medical provider;
determine a first quality metric associated with the medical provider based at least in part on the quality metric data;
determine a difference between the first quality metric and a second quality metric stored in the medical provider account associated with the medical provider; and
perform at least one of:
based at least in part on a determination that the difference is less than a threshold difference, automatically verify the second quality metric associated with the medical provider; or
based at least in part on a determination that the difference is at or above a threshold difference, cause the second quality metric to be manually verified.

20. The one or more computer readable media of claim 19, wherein the quality metric data comprises at least one of:
quality review data associated with services rendered by the medical provider, wherein the quality review data is accumulated by a crowd-sourced review service;
a timeliness metric associated with insurance claims submitted by the medical provider;
an accuracy metric associated with the insurance claims; or
feedback data generated by a patient associated with a service rendered by the medical provider.
